(19) United States
(12) Patent Application Publication
(10) Pub. No.: US 2016/0198975 A1
(43) Pub. Date: Jul. 14, 2016

(54) SYSTEM AND METHOD FOR ANALYSING DATA FROM A MICROWAVE INVERSE SCATTERING APPARATUS

(71) Applicants: David Rhys GIBBINS, Bristtol (GB); Ian James CRADDOCK, Bristol (GB); Tommy Nils Thomas HENRIKSSON, Bristol (GB)

(72) Inventors: David Rhys GIBBINS, Bristtol (GB); Ian James CRADDOCK, Bristol (GB); Tommy Nils Thomas HENRIKSSON, Bristol (GB)

(21) Appl. No.: 14/911,526

(22) PCT Filed: Aug. 14, 2013
(86) PCT No.: PCT/GB2013/052166

§ 371 Date: Feb. 11, 2016

(51) Int. Cl. A61B 5/05; G06K 9/52; A61B 5/00; G06T 7/00

(52) U.S. Cl. CPC A61B 5/0507; G06T 7/0012; G06K 9/52; A61B 5/0035; A61B 5/4312

(57) ABSTRACT

A system for analysing data from a microwave inverse scattering imaging apparatus, said system comprising a processor which is configured to: process data derived from radiation scattered by an object under test by performing a reconstruction process, said reconstruction process being configured to reconstruct the material properties of the object under test by constructing a numerical mode! to fit said data and updating said numerical model in an iterative manner, the processor being further configured to process data concerning information about a feature of interest within the object under test and adapt the reconstruction process by weighting data derived from the scattered radiation on the basis of said information, wherein the weighting selected for a current iteration of the reconstruction process is dependent on the outcome of an earlier iteration.

### FIELD

Embodiments described herein relate generally to the field of Microwave Inverse Scattering.

### BACKGROUND

Microwave inverse scattering (MIS) is an examination and imaging technique which is in principle cheap, completely safe and well-suited to imaging biological tissues and non-destructive testing of non-metallic objects. Reconstructing the material properties of a dielectric object involves repeatedly running full-wave electromagnetic models, comprising a number of transmit antennas and receive antennas. The computational problem scales linearly with the number of transmit antennas and could require days of computation on a multi core 64 bit-desktop PC.

### BRIEF DESCRIPTION OF THE DRAWINGS

Embodiments of the present invention will now be described with reference to the following non-limiting embodiments and examples shown in the below figures in which:

FIG. 1 shows a schematic of a microwave inverse scattering apparatus (MIS);

FIG. 2 is a schematic of an antenna array and an object under test;

FIG. 3 is a flow chart describing a method for constructing a numerical model to model an object under test;

FIG. 4 is a flow chart describing the operation of a system that selects a subset of data for use in the reconstruction process in accordance with an embodiment of the present invention;

FIG. 5 is a schematic of a system of an MIS apparatus in accordance with an embodiment of the present invention;

FIG. 6 is a schematic of an object under test in an MIS apparatus in accordance with an embodiment of the present invention;

FIG. 7 is a flow chart showing a method of determining a perturbation signal which may be performed by a system in accordance with an embodiment of the present invention;

FIG. 8 is a schematic of an object under test;

FIGS. 9(a) and 9(b) are reconstructions of the object under test shown in FIG. 8;

FIG. 10(a) is a plot of the magnitude of the unperturbed signal and the perturbed signal over time; and FIG. 10(b) is a plot of the windowed unperturbed signal derived from FIG. 10(a);

FIG. 11 is a plot of the perturbed signal over time for an object under test;

FIG. 12 is a plot of the perturbed signal of FIG. 11 and the corresponding unperturbed error signal; and

FIG. 13 is a schematic of an object under test to demonstrate the selection of antennas in accordance with an embodiment of the present invention.

### DETAILED DESCRIPTION

Embodiments of the present invention provide a system for a microwave inverse scattering imaging apparatus, said system comprising a processor which is configured to:
process data derived from radiation scattered by an object under test (OUT) by performing a reconstruction process, said reconstruction process being configured to reconstruct the material properties of the OUT by constructing a numerical model to fit said data and updating said numerical model in an iterative manner,
the processor being further configured to process data concerning information about a feature(s) of interest (FOI) within the OUT and adapt the reconstruction process by weighting data derived from the scattered radiation on the basis of said information, wherein the weighting selected for a current iteration of the reconstruction process is dependent on the outcome of an earlier iteration.

In order to identify the FOI the processor may be configured to process a priori information about the feature of interest from at least one source selected from; a previous reconstruction using microwave inverse scattering; or another imaging modality to said microwave inverse scattering apparatus; or a priori knowledge of the OUT.

In one embodiment, the a priori information comprises information relating to the location of a feature of interest of said OUT. However, it may also relate to the composition or size of the FOI in addition to or instead of the location of the feature.

In certain imaging scenarios a great deal is known about the material distribution of the OUT and moreover only certain features, within the OUT, maybe of interest. This fact can exploited by using existing information about the FOI to adapt the reconstruction process on-the-fly to preferentially reconstruct the FOI while reducing computational and time costs and improving the quality of the reconstruction of the FOI.

In an embodiment, during the reconstruction process, the processor is configured to select the weightings, by analysing the results of an earlier iteration with information about the FOI.

In an embodiment, the processor is adapted to:
identify a signature of the feature of interest in the data derived from radiation scattered by said OUT and based on said signature weight the data derived from the scattered radiation.

For example, the signature may be identified by configuring the processor to:
perturb the feature of interest in a numerical model derived from a previous iteration;
simulate data relating to radiation scattered using said perturbed numerical model to produce a perturbed data signal; and
compare said perturbed data signal with the data derived from radiation produced by the numerical model without the perturbation present.

Alternatively, the signature may be identified at a particular iteration by the processor being configured to calculate the radiation pattern of the FOI using the estimate of the material distribution at that iteration as the propagation environment and a numerical or analytical process such as the use of a Green's function or the like.

In one embodiment, the processor may be configured to allocate a weighting of zero to some of the data such that the processor selects some of the data and removes other data from the reconstruction. In other embodiments, the processor is configured to apply a variable weighting such that the influence of data which has been identified as non essential is reduced, but not removed.

The processor is configured to apply said weighting to time domain data, frequency domain data and/or even to the antennas from which the radiation was transmitter and received.

In some embodiments a plurality of antennas will be used where each antenna can function as a transmitting antenna or a receiving antenna. In such embodiments, the apparatus can be configured so that a first antenna is selected as a transmitting antenna and then the remaining antennas are used to receive the scattered radiation. Next the former transmitting antenna is reconfigured as a receiving antenna and one of the former receiving antennas is reconfigured as a transmitting antenna. The data can be collected in this way for a plurality of receiving antennas.

The data from the receiving antennas may be selected/weighted in a number of different ways. A perturbation analysis as described above may be used, in a further embodiment, the data from the transmit antennas may be weighted by calculating the gradients for each transmitter and weighting the data on the basis of said gradients, wherein the volume that contains the OUT is subdivided into voxels, a gradient is calculated for each voxel and each gradient represents the change of the material properties for each voxel calculated during an iteration of the reconstruction process using the data from one transmit antenna.

In a further embodiment, the processor is configured to perform the reconstruction process by calculating the difference between the actual radiation field scattered by the object under test and the estimated radiation field scattered by the numerical estimate of the object, the processor being further configured to measure the progress of the reconstruction by calculating a cost function, the cost function being calculated using the weighted data.

In a further embodiment, the microwave inverse scattering apparatus comprises a plurality of antennas, said antennas being configured to operate as a transmitter and/or a receiver, wherein the processor is configured to perform the reconstruction process by calculating an error signal for each transmitter/receiver pair of antennas and wherein the error signal is calculated using the weighted data.

In the above embodiments, the weighting may be updated for each iteration of the reconstruction process, may be updated every n iterations (where n is an integer of at least 2, or the weighting may be updated based on a measure of the progress of the algorithm.

To summarise, in embodiments, the following data can be weighted to preferentially select:
A subset of the Time domain data
A subset of the Frequency domain data
The transmitting or receiving antennas

The weighting may be based upon:
Apriori Positional/material information from other modalities.
Information from a perturbation analysis using the current reconstruction
Estimated time of flight using current reconstruction
Positional/material information from the current reconstruction
Gradient produced by each transmit antenna.

In further embodiments, a method for analysing data from a microwave inverse scattering imaging apparatus is provided, said MIS imaging apparatus comprising a plurality of antennas which can be configured as either receive or transmit antennas, said apparatus being configured to transmit radiation to an OUT using an antenna configured as a transmit antenna and receive radiation from at least one antenna configured as a receive antenna, said method comprising:
processing data derived from radiation scattered by an OUT by performing a reconstruction process, said reconstruction process being configured to reconstruct the material properties of the OUT by constructing a numerical model to fit said data and updating said numerical model in an iterative manner,
the method further comprising processing data concerning information about a feature of interest within the OUT and adapting the reconstruction process by weighting data derived from the scattered radiation on the basis of said information, wherein the weighting selected for a current iteration of the reconstruction process is dependent on the outcome of an earlier iteration.

Methods in accordance with embodiments of the present invention can be implemented either in hardware or on software in a general purpose computer. Further methods in accordance with embodiments of the present can be implemented in a combination of hardware and software. Methods in accordance with embodiments of the present invention can also be implemented by a single processing apparatus or a distributed network of processing apparatuses.

Since some methods in accordance with embodiments can be implemented by software, some embodiments encompass computer code provided to a general purpose computer on any suitable carrier medium. The carrier medium can comprise any storage medium such as a floppy disk, a CD ROM, a magnetic device or a programmable memory device, or any transient medium such as any signal e.g. an electrical, optical or microwave signal.

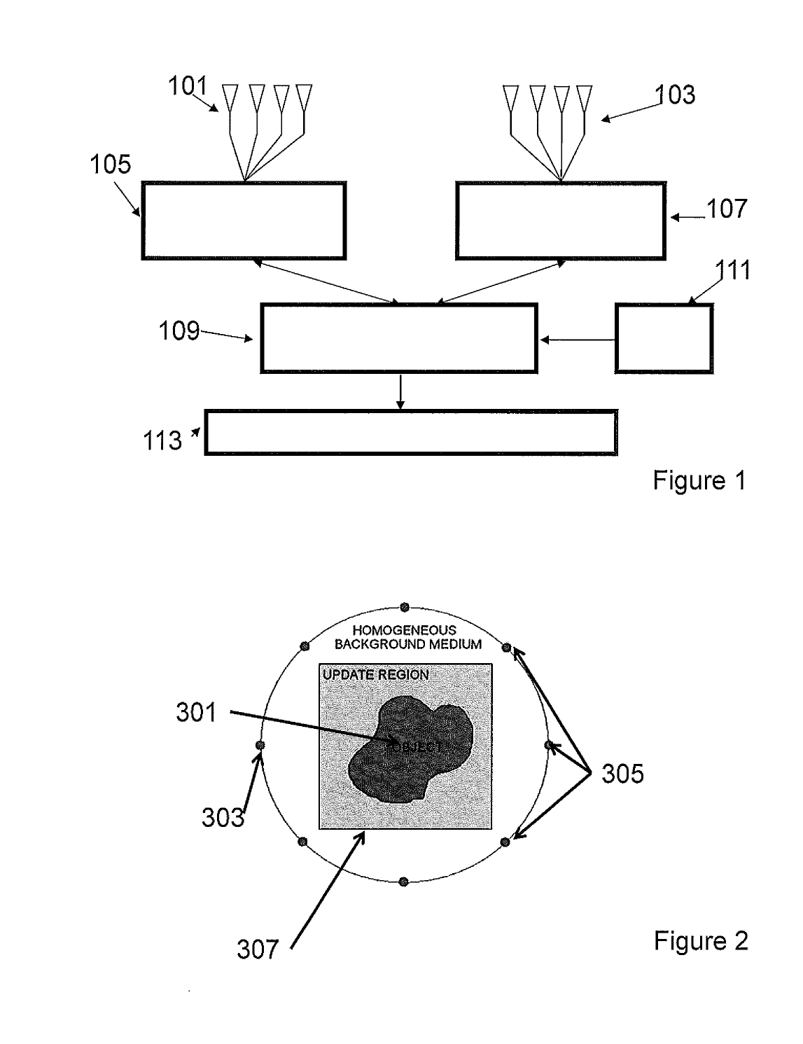

FIG. 1 is a schematic of a medical imaging apparatus for which a microwave system in accordance with an embodiment of the present invention may be used.

The apparatus is a microwave imaging apparatus and comprises a plurality of microwave transmitters 101 and a plurality of microwave receivers 103. It should be noted that the array of transmitters and receivers is shown for illustrative purposes only. It is possible for there to be just a single transmitter 101 which may be stationary or moveable between different positions. Also, it is possible for there to be a single receiver which may be movable between different positions. In an embodiment, the same antenna may act as either a transmitter of a receiver. Thus there may be a plurality of antennas where each antenna may act as a receiver or a transmitter. One of the antennas may be configured to transmit and the other antennas receiver, then a different antenna may be configured to transmit radiation to be received by the other antennas.

The microwave transmitters 101 are controlled by transmitter interface 105. The microwave receivers 103 are controlled by microwave receiver interface 107.

Both the receiver interface 105 and the transmitter interface 107 are in communication with system 109. In some embodiments, the receiver interface 105 and the transmitter interface 107 may be realised by a common component. System 109 is configured to process the data received from receivers 103 in response to emission of radiation by a transmitter from the plurality of transmitters 101.

In one embodiment, the system 109 generates a numerical model of the object under test (OUT) showing the variations in the relative permittivity and the conductivity throughout the object.

In one embodiment, the electromagnetic field data is first modelled using Maxwell's equations.

A cost function is calculated which measures the squared difference between the measured fields and the numerically calculated fields for each transmitter/receiver pair.

The calculated fields may be obtained from a finite difference time domain (FDTD) simulation. The cost function may then be minimized using known techniques. In one embodiment, an iterative approach is used. In a further embodiment, a steepest decent method is used. In further embodiments, Lagrange multipliers were used.

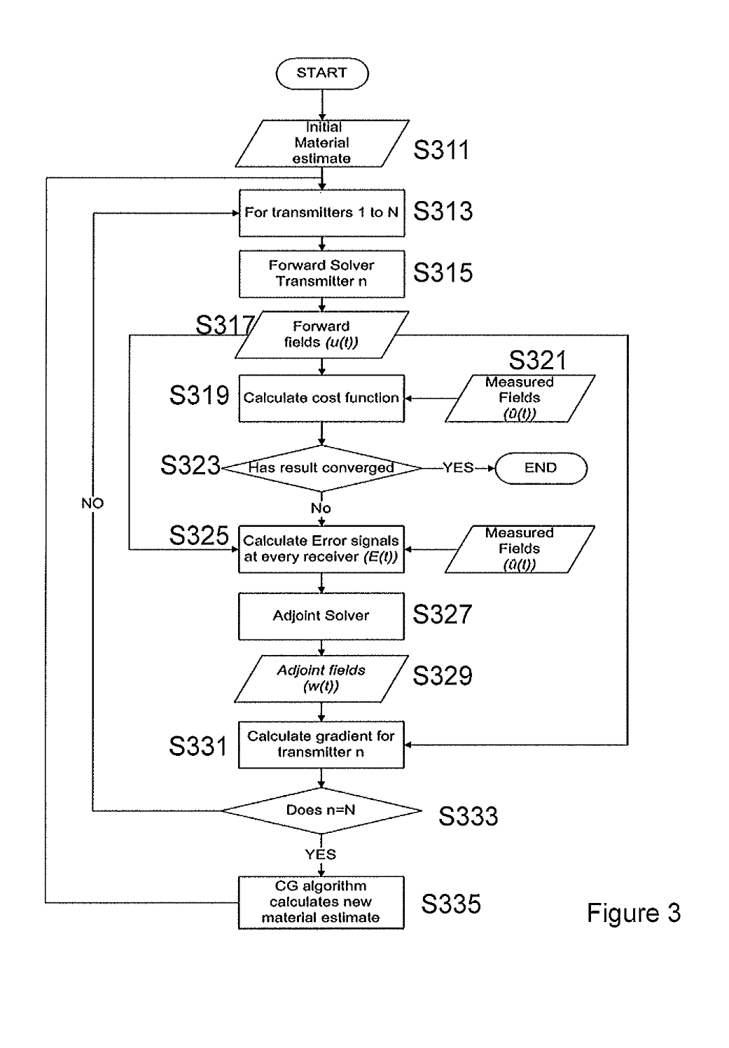

In accordance with a further embodiment, the numerical model is constructed by combining the Forward/Back ward Time Stepping (FBTS) inverse algorithm with a non-linear Conjugate Gradient optimisation. A flow chart of the reconstruction process is shown in FIG. 3.

As shown in FIG. 2, an unknown target 301 is illuminated with microwave radiation radiated by an antenna 303 that is part of an array 305 surrounding the target 301. The scattered fields are recorded by the other antennas 305 in the array. Once complete the antenna 303 in the transmission mode changes and the process repeats for all required transmitters. The recorded information is known as the measured fields.

An initial estimate of the properties of the target is made, this is descritised and a numerical model produced. The numerical model is used to simulate the target in FIG. 2. This is the starting point S311 in the flowchart of FIG. 3. In an embodiment, the initial estimate is determined from basic information concerning the OUT. For example, if the object under test is the human breast, the initial estimate may comprise some details of the basic shape and typical average material properties for a breast.

The initial estimate can be made by physically measuring the outer surface of the OUT—this has been done in a number of different ways e.g. scanning laser, microwave radar. A guess of the average material properties can be made based on what is typical. E.g. in the case of a post-menopausal breast that is known to consist of mainly adipose tissue the corresponding tissue properties could be used alternatively an average value could be obtained via measurement.

In step S313, for each transmitter used, this model is fed into a finite difference time domain (FDTD) electromagnetic solver and is interrogated with microwaves in the same manner as that used to illuminate the measured case. The fields are once again recorded at the receivers but also at every voxel within the update region 307 (FIG. 2). This is known as the forward solver S315 and the recorded data is known as the forward fields S317.

The measured and estimated fields at the receivers are used to quantify the difference between the actual and estimated material distributions in a scalar value using the cost function J(p) in step S319:

$$J ( p ) = {{\int }_{0}}^{T} {\sum }_{i = 1}^{I} {\sum }_{m = 1}^{M} {\vert {u}_{i , m , p} - {\hat{u}}_{i , m} \vert}^{2} d t$$

Where u_{i,m,p} are the estimated fields at receiver m produced by transmitter i for parameter estimate p and û_{i,m} are the equivalent measured fields 8321. At this stage, the process is checked against a convergence criteria in step S323.

An error signal is calculated for each transmitter/receiver pair by subtracting the time-domain measured fields from the estimated fields in step S325.

In step S327, this error signal is reversed in time, fed back into the FDTD solver and re-radiated back into the volume simultaneously from every receiver, using the same material estimate as for the forward case. The resulting fields are again recorded in the reconstruction volume. This is known as the adjoint or backwards solver and the fields are known as the adjoint fields S329.

In step S331, at each point in the update region a material update is calculated using the forward and adjoint fields. The material update is calculated in the form of a gradient which indicates the direction in which the material properties within each voxel must be changed in order to minimise the cost function and improve the material estimate. If it is assumed that the dielectric material properties of the OUT are non-dispersive then they maybe described using the conductivity form of frequency domain complex permittivity ∈(ω):

$$ɛ ( ω ) = {ɛ}^{′} {ɛ}_{0} - \frac{σ}{jω}$$

Where ∈′ is the relative real part of the permittivity, ∈₀ is the permittivity of free space and is constant, σ is the conductivity, ω is the angular frequency and j=√(−1). To reconstruct the object, gradients are calculated for ∈′(G_{∈′}(r)) and σ(G_{σ}(r)) at location r, using the FBTS equations:

$${G}_{{ɛ}^{′}} ( r ) = - {\sum }_{i = 1}^{I} {{\int }_{0}}^{T} {w}_{i} ( r , t ) \frac{\partial {u}_{i} ( r , t )}{\partial t} d t$$

$${G}_{σ} ( r ) = - {\sum }_{i = 1}^{I} {{\int }_{0}}^{T} {w}_{i} ( r , t ) \partial {u}_{i} ( r , t ) d t$$

Where I is the field component specifier, wᵢ(r, t) is the ith adjoint field value at point r at time t and uᵢ(r, t) is the equivalent forward field component. In step S333 a check is performed to see if the gradients need to be calculated for further transmit antennas. Gradients are then calculated for each transmit antenna and are then summed to produce a single gradient that is used to calculate a new material estimate in step S335.

The gradients obtained via this process are relative. In the first instance a line search is used to find the scaling factor for the parameter update that minimises the cost function Fp. This scaled material update is then used as a starting point for an iterative procedure based on the non linear Polak-Ribière/conjugate gradient optimisation technique. The iterative procedure is halted once a pre-determined convergence criterion is reached in step S323.

In an embodiment, the above process is implemented using Lagrange multipliers

Variations on this approach could replace the above conjugate gradient algorithm with a different method to guide the iterative process, examples being the steepest descent or Quasi-Newton methods. In certain scenarios it maybe necessary to use a dispersive model rather than using the conductivity model to describe the complex permittivity. This is exemplified by the Deybe form of complex permittivity:

$$ɛ ( ω ) = {ɛ}_{∞} {ɛ}_{0} - \frac{{ɛ}_{0} ( {ɛ}_{s} - {ɛ}_{∞} )}{1 - jωτ} - \frac{σ}{jω}$$

Where ∈_{∞} is the relative permittivity at infinite frequency, ∈ₛ is the static permittivity and τ is the relaxation time. In this case the MIS algorithm would solve for σ, ∈_{∞} and ∈ₛ and appropriate gradients would be calculated at each iteration, otherwise the solver would be unchanged. An alternative implementation could use a Frequency domain MIS solver using time domain EM-simulator to calculate the scattered fields which are then Fourier transformed to obtain the frequency domain fields.

Reconstructing the OUT in this way can require considerable computation resources.

In the embodiment shown in FIG. 1, unit 111 provides an input to system 109. Unit 111 provides the system 109 with information about the OUT. The data is provided in such a way that it allows the system 109 to reduce the amount of data derived from the transmit antennas 101 and the receive antennas 103 such that the computational burden of performing the numerical analysis is reduced. FIGS. 6 to 13 will describe examples of the type of information which unit 111 provides to system 109. The action of unit 111 will, in some embodiments, cause system 109 to ignore certain data and process only data contained in a small window of the received radiation data. In other cases, it will cause the system 109 to control the transmitter interface 105 and/or the receiver interface 107 to only use certain transmit antennas 101 and/or the receive antennas 103.

The system outputs the numerical model to output unit 113. Output unit 113 may give pictorial output of the OUT and/or may give certain detail numerical information about the physical properties of the object.

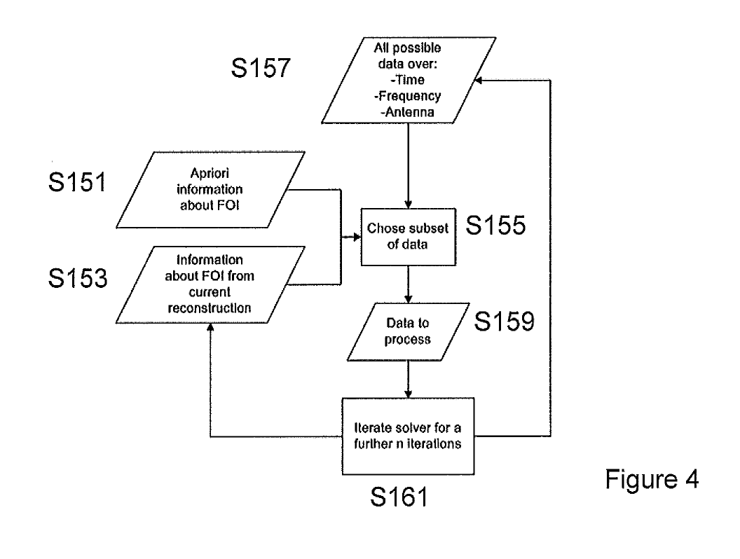

To clarify the procedure, FIG. 4 shows the basic steps.

In steps S151 and/or S153, the system receives information concerning the FOI during a particular iteration of the solver. In step S151 this information originates a priori to the reconstruction process in step S161. In one embodiment this information is acquired using a different imaging modality, for example, an MRI scan, an X-ray scan etc. In step S153 this information originates from a previous step in the reconstruction process. In one embodiment, this information relates to position and/or size of specific FOI within the object. In further embodiments, this may be information relating to the specific frequencies which should be examined or the specific parts of a received time domain signal which should be processed in order to generate the numerical model.

In step S155 the information in steps S151 and S153 is used to select the data of interest 5159 in step S155. The data is selected from the whole dataset collected. The selected data will then be used in the reconstruction process of S161. The reconstruction process S161 has been explained with reference to FIG. 3.

The data is selected by applying a weighting such that the influence of data which is considered to be of lower importance to the reconstruction of the FOI can be reduced. In some embodiments, some of the weights are set to zero so that certain data is completely removed from the reconstruction process. In other embodiments, the weighting is set to simply reduce the influence of some data. This could be in the form of a square window or a Tukey window that smooths the transition from the data of interest to that to be suppressed.

In some embodiments, selection of the radiation may be achieved at step S313 of the reconstruction process (FIG. 3) through the use of a subset of the transmit antennas used in the measurement of the OUT. In addition at step S315 (FIG. 3) various properties of the radiation transmitted by these antennas may be selected such as frequency content, pulse type etc. The transmitters selected may be determined by the gradients calculated in step S331 (FIG. 3) or by choosing only those transmit antennas which prove to be, by some metric, most relevant to reconstructing a particular feature of the OUT.

In another embodiment the radiation maybe selected at step S325 (FIG. 3) by choosing only those receive antennas which prove to be, by some metric, most relevant to reconstructing a particular feature of the OUT.

The data may also be selected from time of flight data from the current reconstruction and/or based on the estimated material properties.

In further embodiments selection of the radiation of interest can be implemented at steps S319 and S325 (FIG. 3) to calculate the cost function or error signals and involves the system 109 selecting which radiation to process from the received radiation.

After the reconstruction process S161 is carried out using the selected data, a new numerical material estimated is generated in step S157 as previously explained. This new numerical material estimate then forms the updated numerical model to be supplied to the FDTD solver for the next iteration.

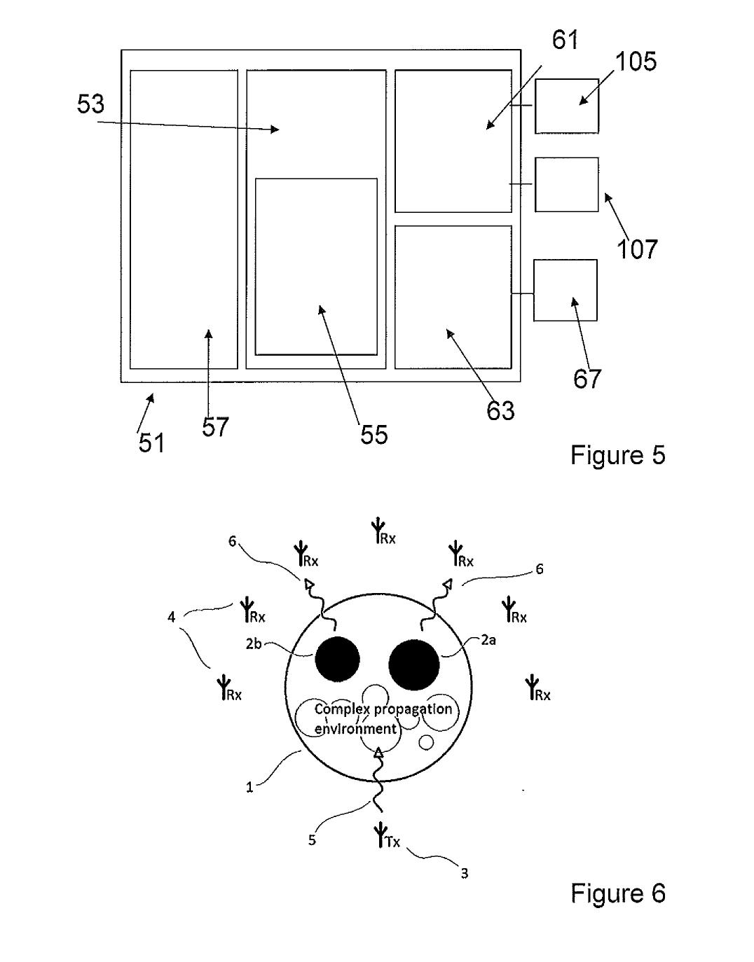

For completeness, FIG. 5 shows a schematic of the system 109. The system 109 comprises a processor 53 which executes a programme 55. The system 109 further comprises storage or memory 57. Storage 57 stores data which is used by the programme 55 in order to construct the numerical model. In one embodiment, the data received from unit 111 is stored in memory 57.

The system comprises transmission/reception module 61 which controls the transmission interface 105 and the reception interface 107. As previously noted, the transmission and reception interfaces may be a common interface.

Output module 63 modifies the data output from programme 53 in a form that it can be displayed on output 113.

The apparatus described above with reference to FIGS. 1 to 6 allows a reconstruction of a FOI within an OUT using properties of the FOI which are known. The information about the FOI may be known from an earlier investigation such as a scan by an alternative modality, an earlier reconstruction process etc. The information about the FOI is compared with results from an earlier iteration such used in such a way that it allows the computational requirements to be relaxed and also can increase the sensitivity of the reconstruction. As the reconstruction progresses, the results of the later iteration can be compared with the known information about the FOI such that the weightings can be updated as the reconstruction progresses.

As mentioned above, a priori information can be obtained from an alternative modality or a previous scan using this modality to adapt the reconstruction process from the start. By monitoring the state of the material estimate of the OUT as the reconstruction progresses and tailoring the reconstruction process during the generation of the numerical model, the reconstruction process can be continually adapted to use the data which has been identified as giving the most pertinent information regarding the FOI.

In one embodiment, the data is selected for each iteration of the reconstruction process. In a further embodiment, the selected data/weightings are not updated for every iteration, but may be updated for every n iterations, where n is an integer of at least 2. In a yet further embodiment, the selected data/weightings may be updated based on a measure of the reconstruction process. For example, if the material estimate (and so the propagation environment used to obtain the FOI signature) changes by a certain amount then the selected data/weightings may be updated at the next iteration.

FIG. 6 shows a schematic of an OUT 1 where it is known that there are two FOIs 2a and 2b. The 2 FOIs, whose rough positions are known, are within a complex heterogeneous object and whose exact position, size and material properties are the object of the reconstruction. The object is irradiated with radiation from single transmission antenna 3, the transmitted radiation 5 is scattered by OUT 1 and collected as scattered radiation 6 by array of receiving antennas 4.

As can be seen from FIG. 6 in wide-bandwidth time domain microwave inverse scattering, when the OUT is interrogated with a very short nano-second pulse, only a certain portion of the scattered fields recorded at the receiving antennas 4 will contain information about the FOIs 2a and 2b. In an embodiment, a frequency range of 0.5 GHz to 10 GHz is used and pulse widths of the order of 3.5 to 0.2 ns.

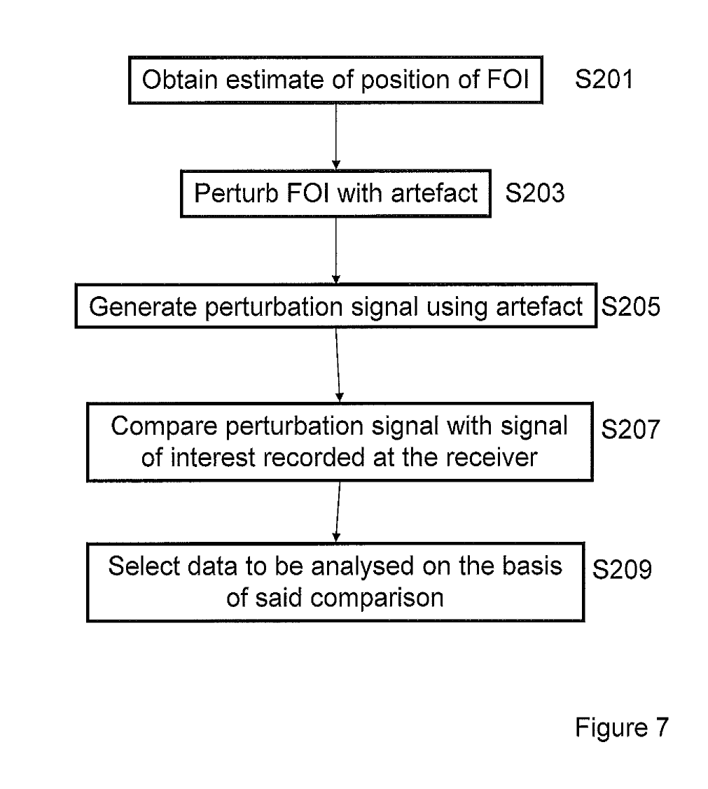

The part of the signal containing information about the FOI may be identified by calculating the approximate time of flight from the transmitting antenna 3 via the FOI to the receiving antennas 4 using the material estimate at the current iteration of the solver. Alternatively the part of the signal may be identified by perturbation analysis which will be described with reference to FIG. 7.

Once the approximate position of the FOI has been identified, an additional Forward EM-simulation is run in which its positional properties are manually perturbed.

In step S201, a position of the features in the FOI is estimated. This may be done by using a scan from another modality e.g. X-ray. MRI, some other type of prior knowledge of the OUT or from earlier scans.

In an embodiment, at each receive antenna in the array, a perturbation signal is calculated in the following way:

P(t)=u(t)_{pb}−u(t) equation 1

Where P(t) is the time domain perturbation signal u(t)_{pb} is the total signal at the receiver recorded from the forward FDTD model with the perturbation object is present and u(t) is the total signal recorded at the receiver from the forward FDTD model without the perturbation present.

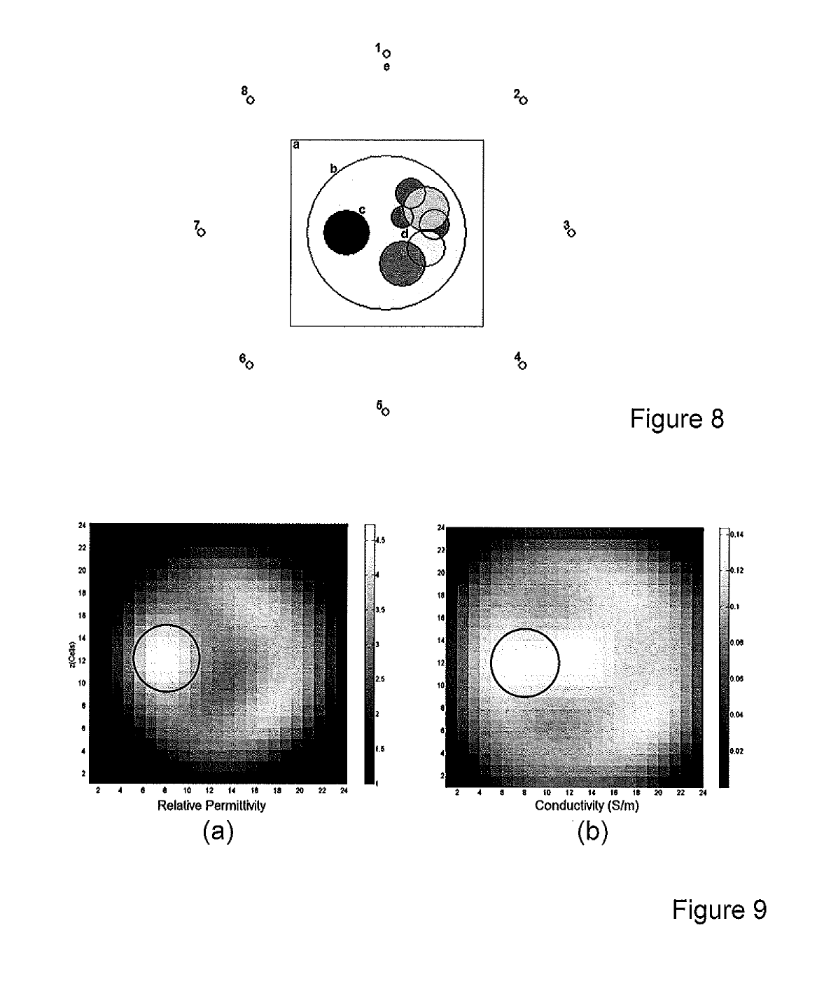

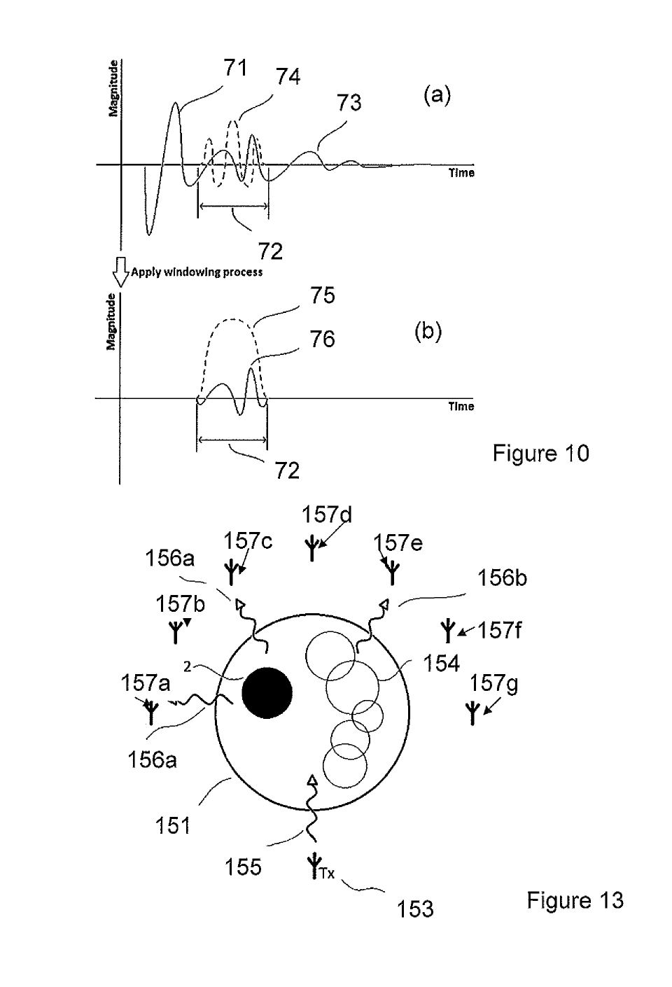

As an example, FIG. 8 shows a spherical FOI c embedded in an OUT with a number of other objects identified as d. FIG. 9(a) shows a reconstruction of the OUT after 20 iterations. The position of the FOI is shown by a black circle in both FIG. 10(a) which shows the relative permittivity and FIG. 9(b) which shows the conductivity. However, the material values of the object have not been reconstructed.

To find the part of the time domain signal that contains information about the FOI, the current numerical estimate of the OUT is perturbed by the addition of an object in the position of the FOI in step S203.

In the current example, the FOI is perturbed by the addition of an object with small dielectric values (relative permittivity equals 0.5, conductivity equals 0.05 s/m) that is the same size and in the same location as the FOI.

In step S205, a perturbed signal is then generated at the receiver, the signal models what the scattered radiation would look like if the OUT contained the perturbed object. The perturbation signal is then calculated using equation 1. The perturbation signal represents the additional scattered fields that result from the introduction of the FOI

In step 207 the perturbed signal is then compared with the signal recorded at the receiver in the unperturbed case. This may be done in a number of ways.

In one embodiment, the perturbed signal P(t) can be compared with any time domain signal recorded at that receiver (as shown in FIG. 10(a)) where the unperturbed u(t) signal 71 is compared with the perturbation signal P(t) 74, and the perturbation signal 74 is used to window 72 the unperturbed signal to produce isolated signal 76 of FIG. 10(b).

In step S209, this can then be used to select data by modifying the calculation of the cost function by windowing the forward u(t) and measured û(t) signals recorded at the receivers. This is done by applying limits to the time integration in the cost function calculation:

$$J ( p ) = {{\int }_{{T}_{1}}}^{{T}_{2}} {\sum }_{i = 1}^{I} {\sum }_{m = 1}^{M} {\vert {u}_{i , m , p} - {\hat{u}}_{i , m} \vert}^{2} d t$$

Where the limits of the integration T₁ are T₂ the start and stop limits or me time window. This effectively applies a square filter to the data.

Windowing the error signals E(t) recorded at the receivers, using a window defined by P(t) can also be beneficial and result in preferential reconstruction of the FOI. The error signals are calculated as:

E(t)=u(t)−\hat{u}(t)

Where u(t) are the time-domain forward signals at the receivers, obtained from the estimated FDTD model and û(t) are the total time domain signals obtained by direct measurement of the OUT. These are the measured and forward fields at step S325 in the algorithm description above in FIG. 3. Later, in FIG. 12 P(t) is compared with the associated error signal.

As stated in the explanation of the algorithm above, the error signals are reradiated by the antenna in the adjoint simulation to obtain the adjoint fields in the update domain. These are then used to calculate the gradient, so applying the window to the error signal will limit the signal being re-radiated to those parts which contain information about the FOI, preferentially reconstructing the FOI over everything else.

In an embodiment, the window used may be a straight square window where just the part of the signal identified by the perturbation analysis would be used to calculate the cost function.

In an embodiment, when calculating the window function to be used for windowing error signals, a window function is selected with smooth edges as shown as 75 in FIG. 10(b) as the smooth edges reduce the noise produced by applying the window. Such filters may include, a “Tukey window” or “Plank Taper window”.

When windowing with the perturbation signal, the position of the window is approximate because the material properties of the model are not yet fully reconstructed and so time of flight will be different to the final model. In an embodiment, the position and/or size of the windows are recalculated in an iterative manner as the calculation proceeds.

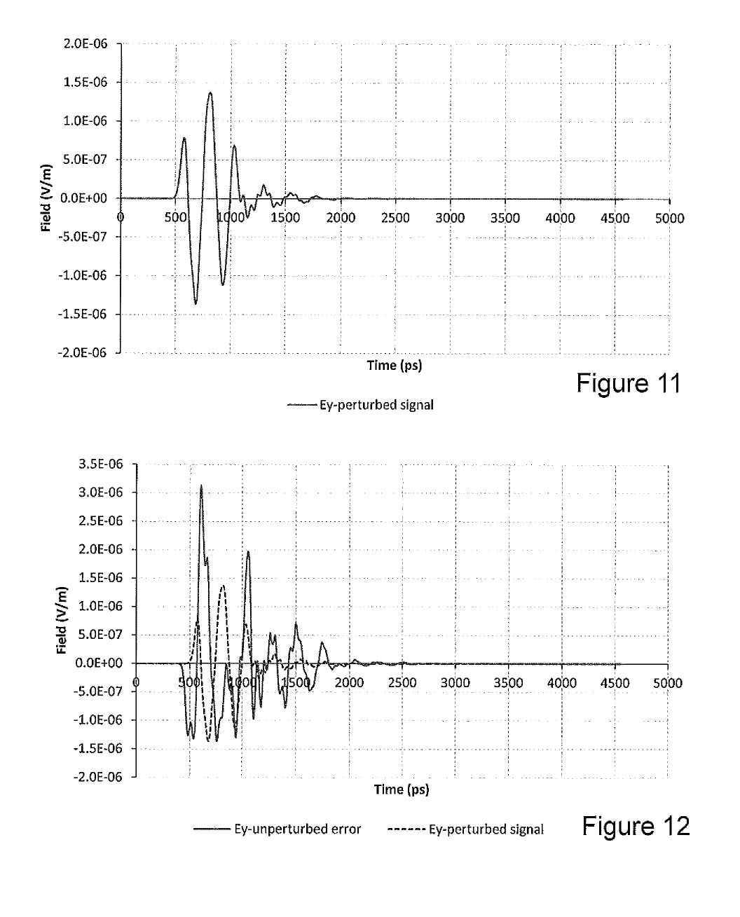

FIG. 11 shows the perturbation signal P(t), generated from applying the perturbation object in the above analysis.

FIG. 12 compares this perturbation signal with the unperturbed error signal from the above described analysis. It can be seen that the perturbed signal occupies a sub region of the full signal. It is this region that is crucial to reconstruction in order to determine information about the FOI. Therefore by performing this analysis, the error signal in the region of the FOI is preferentially reconstructed.

The above has described a selection of data using a time domain signal. However, other options are possible.

In a further embodiment, the windowing approach is applied to the frequency domain. Here, once a spectrum that contains information about the FOI is identified, this can be used to set a maximum bound for the frequency content of an interrogating signal and/or if a frequency hopping approach is required, how large that frequency hop needs to be.

Selecting certain frequencies may also comprise the use of frequency domain filters to the waveforms recorded at the receivers and/or 3D field “snap shots”, which are integral to the reconstruction process. Here, the snapshots record the fields within the update region of FIG. 2 in both the forward and backward runs (uᵢ(r, t)—forward fields and wᵢ(r,t)—the adjoint fields) that are used to calculate the material gradients at each point in the update region.

By suppressing parts of the frequency spectrum that don't contain information about the FOI, the FOI will be preferentially reconstructed while sources of noise will be removed.

The frequency domain can be processed in a similar manner to that described with reference FIGS. 7 to 12 for the time domain.

A perturbation signal can be constructed using an artefact in the position of the FOI. The position of the artefact can be identified in the same manner as described for the time domain situation.

In a further embodiment, the radiation to be selected for the reconstruction is selected on the basis of those receiving antennas whose received signals contain the highest proportion of information about the FOI.

FIG. 13 shows an OUT with an FOI 152. Radiation is transmitted from transmitter 153. In addition to the FOI 152, the OUT 151 contains other unknown objects 154.

As can be seen in FIG. 13, the scattered radiation 156a and 156b will be detected by receiving antennas 157a to 157g. Antennas 157a to 157c are more likely to receive the radiation 156a which has been scattered by FOI 152. Whereas antennas 157d to 157g are more likely to receive the radiation 156b that has been scattered by unknown objects 154.

In one embodiment, the antennas which are chosen may be chosen in a geometric manner by choosing those antennas that are closer to or on the same side of the object as the FOI. However, the approach discussed in relation to FIG. 7 may also be used.

In an embodiment, in step S207, the perturbation signal P(t) is compared with the unperturbed signal u(t) by calculating a ratio:

$$R = \frac{{\int }_{0}^{T} P ( t ) d t}{{\int }_{0}^{T} u ( t ) d t}$$

This allows the received perturbation signal to be scaled using the overall signal (u(t)). This relates the magnitude of P(t) to the rest of the signal and so gives a measure of the proportion of information contained in u(t) that pertains to the FOI. Receive antennas can then be rated and chosen based on this metric.

It is also possible to select the transmitter using a similar approach. In this case a metric is obtained for each transmitting antenna by summing the ratios for the N antennas acting as receivers. So for transmitter q, R_{q} would be calculated as:

$${R}_{q} = {\sum }_{n = 1}^{N} \frac{{\int }_{0}^{T} {P ( t )}_{n} d t}{{\int }_{0}^{T} {u ( t )}_{n} d t}$$

Transmit antennas can then be rated and chosen based on this metric.

When choosing both transmit and receive antennas there are other possible metrics that could be used including just using the energy in P(t) or calculating the ratio between P(t) and E(t) the error signal.

The size of this set of antennas can be chosen an arbitrary limitation, e.g. the 2 receive antennas with the highest ratio will be chosen. The actual set number selected could be determined by the known amount of computing resources available. Alternatively, the antennas may be selected by choosing antennas whose ratio is above a certain, threshold value.

In another embodiment the sub set of transmitters can be selected by examining the gradient produced by that transmit antenna at step S331 (FIG. 3). In an embodiment, a gradient is calculated for each voxel as described above. A gradient can be calculated for a transmit antenna at an identified voxel of interest, for example a voxel in the FOI. In a further embodiment, the gradient can be calculated over a larger area by averaging the gradients over the voxels in an area.

In an embodiment, the FOI can be identified using a pattern recognition algorithm.

The gradient produced by each transmit antenna can be compared to the known properties of the FOI and those antennas whose gradients are deemed, by some metric, to contribute positively to the reconstruction of the FOI will be selected, while those that do not are suppressed. For example, if a gradient indicates that the numerical model will be updated to a value which is close to an expected value for the FOI, the transmitter antenna which produced that gradient will be allocated a greater weighting.

The selected gradients are then used to produce the material update for the next iteration of the solver in step S335 and the selected sub set of antennas are used in subsequent iterations of the reconstruction process. This process may repeated at selected intervals to adapt to the changing nature of the reconstruction process.

The comparison of the known properties of the FOI and the gradient produced by each antenna could be carried out in a number of ways including but not exclusively; examining the shape of features within the gradient examining, examining the numerical values of features within the gradient or comparing the second moment of area.

The methods in accordance with the above embodiment preferentially reconstruct the image in one specific area of the OUT. Thus, the reconstruction in that area will converge faster with fewer simulations resulting in shorter run times and subsequently lower computational costs. Further, the cost function which is generated is more sensitive to the reconstruction of the FOI. The application of the windowing techniques will reduce the amount of noise in the reconstruction signal resulting in more accurate reconstructions.

While certain embodiments have been described, these embodiments have been presented by way of example only, and are not intended to limit the scope of the inventions. Indeed, the novel methods and systems described herein may be embodied in a variety of other forms; furthermore, various omission, substitutions and changes in the form of the methods and systems described herein may be made without departing from the spirit of the inventions. The accompanying claims and their equivalents are intended to cover such form or modifications as would fall within the scope and spirit of the inventions.

1. A system for analysing data from a microwave inverse scattering imaging apparatus, said system comprising a processor which is configured to:
process data derived from radiation scattered by an object under test by performing a reconstruction process, said reconstruction process being configured to reconstruct the material properties of the object under test by constructing a numerical model to fit said data and updating said numerical model in an iterative manner,
the processor being further configured to process data concerning information about a feature of interest within the object under test and adapt the reconstruction process by weighting data derived from the scattered radiation on the basis of said information, wherein the weighting selected for a current iteration of the reconstruction process is dependent on the outcome of an earlier iteration.

2. A system according to claim 1, wherein the processor is configured to receive information about the feature of interest from at least one source selected from: a previous reconstruction using microwave inverse scattering; another imaging modality to said microwave inverse scattering apparatus; or a priori knowledge of the object under test.

3. A system according to claim 1, wherein the processor is configured to select the weightings, by analysing the results of an earlier iteration with information about the feature of interest.

4. A system according to claim 1, wherein said processor is adapted to:
identify a signature of the feature of interest in the data derived from radiation scattered by said object under test and based on said signature weight the data derived from the scattered radiation.

5. A system according to claim 4, wherein the signature is identified by the processor being configured to:
perturb the feature of interest in a numerical model derived from a previous iteration;
simulate data relating to radiation scattered using said perturbed numerical model to produce a perturbed data signal; and
compare said perturbed data signal with the data derived from radiation produced by the numerical model without the perturbation present.

6. A system according to claim 4, wherein the signature is identified by the processor being configured to calculate the radiation pattern of the feature of interest.

7. A system according to claim 6, wherein the processor is configured to calculate the radiation pattern using a Green's function.

8. A system according to claim 1, wherein the processor is configured to allocate a weighting of zero to some of the data.

9. A system according to claim 1, wherein the processor is configured to apply said weighting to time domain data.

10. A system according to claim 1, wherein the processor is configured to apply said weighting to frequency domain data.

11. A system according to claim 1, wherein the microwave inverse scattering system comprises a plurality of antennas, for receiving radiation scattered by the object, the processor being configured to apply said weighting to data on the basis of the antenna at which the data is received.

12. A system according to claim 1, wherein the microwave inverse scattering apparatus comprises a plurality of antennas, the antennas being configured to transmit radiation, the processor being configured to apply said weighting to data on the basis of the transmitting antenna from which it originates.

13. A system according to claim 12, wherein the processor is configured to apply said weighting by calculating the gradients for each transmitter and weighting the data on the basis of said gradients, wherein a volume that contains the object under test is subdivided into voxels, a gradient is calculated for each voxel and each gradient represents the change of the material properties for each voxel calculated during an iteration of the reconstruction process using the data from one transmit antenna.

14. A system according to claim 1, wherein the processor is configured to perform the reconstruction process by calculating the difference between the actual radiation field scattered by the object under test and the estimated radiation field scattered by the numerical estimate of the object, the processor being further configured to measure the progress of the reconstruction by calculating a cost function, the cost function being calculated using the weighted data.

15. A system according to claim 1, wherein the microwave inverse scattering apparatus comprises a plurality of antennas, said antennas being configured to operate as a transmitter and/or a receiver, wherein the processor is configured to perform the reconstruction process by calculating an error signal for each transmitter/receiver pair of antennas and wherein the error signal is calculated using the weighted data.

16. A system according to claim 1, wherein the weighting is updated for each iteration of the reconstruction process.

17. A microwave inverse scattering apparatus comprising a plurality of antennas which can be configured as either receive or transmit antennas and a system as recited in claim 1.

18. A method for analysing data from a microwave inverse scattering imaging apparatus, said microwave inverse scattering imaging apparatus comprising a plurality of antennas which can be configured as either receive or transmit antennas, said apparatus being configured to transmit radiation to an object under test using an antenna configured as a transmit antenna and receive radiation from at least one antenna configured as a receive antenna, said method comprising:
processing data derived from radiation scattered by an object under test by performing a reconstruction process, said reconstruction process being configured to reconstruct the material properties of the object under test by constructing a numerical model to fit said data and updating said numerical model in an iterative manner,
the method further comprising processing data concerning information about a feature of interest within the object under test and adapting the reconstruction process by weighting data derived from the scattered radiation on the basis of said information, wherein the weighting selected for a current iteration of the reconstruction process is dependent on the outcome of an earlier iteration.

19. A carrier medium comprising computer readable code configured to cause a computer to perform the method of claim 18.
